(19) United States
(12) Patent Application Publication
(10) Pub. No.: US 2004/0048258 A1
(43) Pub. Date: Mar. 11, 2004

(54) Multiple-gene diagnostic probes and assay kits and method for the assessment of multiple markers for breast cancer prognosis

(75) Inventors: Richard Idem Somiari, Johnstown, PA (US); Stella Bassey Somiari, Johnstown, PA (US); F. Nicholas Jacobs, Davidsville, PA (US); Rick Jordan, Johnstown, PA (US)

Correspondence Address: STEPTOE & JOHNSON LLP, 1330 Connecticut Ave., NW, Washington, DC, 20036, US

(73) Assignee: WINDBER RESEARCH INSTITUTE, Windber, PA

(21) Appl. No.: 10/237,614

(22) Filed: Sep. 10, 2002

(51) Int. Cl. C12Q 1/68

(52) U.S. Cl. 435/6

(57) ABSTRACT

The problem of inadequate broad-range prognostic factors for breast cancer is solved by providing a multi-gene probe for a single-step determination of disease outcome. The 5-genes chosen (HER2, Topo IIα, NM23-H1, CK19 and MMP9) are known to show altered expression in different breast tumors. The altered expression of any one of them has a similar prognostic significance, with respect to disease-free survival or overall survival. The multi-gene probe is labeled and used to screen a tumor specimen. The signal generated after the assay provides information that has prognostic significance. Since five prognostic markers are simultaneously assessed, a wider variety of breast cancers are covered and disease outcome prediction is improved in a wider population.

### STATEMENT REGARDING FEDERALLY SPONSORED RESEARCH OR DEVELOPMENT

This invention was made with government support under the Clinical Breast Care Project, Contract Number 17330, awarded by the Henry M. Jackson Foundation For The Advancement Of Military Medicine as a subcontract under Grant Number MDA 905-00-1-0022 from the Uniformed Services University of the Health Sciences.

### BACKGROUND OF THE INVENTION

1. Field of the Invention

The present invention relates to multiple diagnostic probes for the assessment of multiple markers for breast cancer prognosis. The invention also relates to assay kits containing multi-gene probes. In addition, the invention relates to a method for the assessment of multiple markers for breast cancer prognosis.

2. Description of the Related Art

Cancer is a disease that results when the controls that regulate normal cell growth break down. The growth and development of normal cells are subject to a multitude of different types of control. A fully malignant cancer cell appears to have lost most, if not all, of these controls. However, conditions that seem to represent intermediate stages, when only some of the controls have been disrupted, can be detected. Thus, the progression from a normal cell to a malignant cell is a multistep process, each step corresponding to the breakdown of a normal cellular control mechanism.

Normal growth controls appear to become ineffective because of mutations in the cellular genes coding for components of the regulatory mechanism. Cancer can therefore result from the accumulation of a series of specific mutations in the malignant cell.

Oncogenes are genes whose expression causes cells to become cancerous. The normal version of the gene (termed a proto-oncogene) becomes mutated so that it is overactive. Because of their overactivity, oncogenes are genetically dominant over proto-oncogenes, that is only one copy of an oncogene is sufficient to cause a change in the cell's behavior.

The oncogene differs from the normal proto-oncogene in important ways. The coding function of the gene may be unaltered but may be transcribed at a higher rate or under different circumstances from normal. This results in overproduction of a normal gene product. Under other circumstances, there may be under-production of a normal gene product.

Tumor suppressor genes act in a fundamentally different way from oncogenes. Whereas proto-oncogenes are converted to oncogenes by mutations that increase the genes' activity, tumor suppressor genes become oncogenic as the result of mutations that eliminate their normal activity. The normal, unmutated version of a tumor suppressor gene acts to inhibit a normal cell from entering mitosis and cell division. Removal of this negative control allows a cell to divide.

Amplification, overexpression and/or underexpression of some proto-oncogenes and tumor suppressor genes are useful clinically for breast cancer prognosis. However, there is currently no single strong independent marker that is useful for predicting disease outcome in a majority of patients.

The number of clinical laboratory assays currently used in oncology is very small. For breast cancer, only three, namely, estrogen, progesterone and HER2 status are currently assessed. Unfortunately, these three only provide useful prognostic and predictive information in a small number of patients. For example, although HER2 (a proto-oncogene) has emerged as a strong independent prognostic and predictive marker for breast cancer, it is only useful clinically in about 25 to 30% of cases. In the last five years, the College of American Pathologists, the American Society of Clinical Oncology expert panels and the Joint Committee on Cancer have carefully considered many markers proposed for managing breast cancer but have found none with proven clinical utility sufficient to justify their adoption for routine practice and no single marker has been found which gives a consistent result in all manifestations.

There are ongoing studies aimed at identifying new and broad-spectrum markers that will be useful in many cases. But it is unlikely that a single broad-range marker will be found since multiple biochemical pathways are associated with the onset, progression and/or severity of breast cancer. Moreover, the relevant pathways involved may be different in different individuals due to other compounding factors such as aging, race, nutrition, habit and environment.

An object of the present invention is to provide a more sensitive, relatively faster and cost effective approach for assessing the status of prognostic markers in breast cancer. This and other objects of the present invention will become more apparent from a consideration of the following description and claims.

### BRIEF SUMMARY OF THE INVENTION

The present invention involves the use of a multiple-gene diagnostic probe targeting the HER2, Topo IIα, NM23-H1, CK19 and MMP9 genes. The invention permits a simultaneous assessment of at least five specific but independent changes in DNA number that enable prognosis. All these changes do not necessarily occur together always or in sequence in each patient. However, because one detectable change (at least) will occur in greater than 95% of cancers, the invention provides a one-step assessment of the molecular status of breast tumors. The present innovation is more sensitive and improves the likelihood of detecting molecular changes of prognostic significance in a larger patient population at a relatively lower cost.

The basic elements of the multi-gene probe of the invention are the following:

i. labeled fragments complementary to multiple but unique regions of the HER2 gene sequence;

ii. labeled fragments complementary to multiple but unique regions of the Topo IIα gene sequence;

iii. labeled fragments complementary to multiple but unique regions of the NM23-H1 gene sequence;

iv. a labeled fragment complementary to the CK19 gene sequence; and

v. labeled fragments complementary to at least a region of the MMP9 gene sequence; each of the fragments being labeled with a different label and each of the fragments being useable to detect its complementary gene sequence by hybridization. The multi-gene probe may be packaged in the form of an assay kit.

The basic elements of the method of making the multi-gene probe of the invention are the following:

i. generation of gene-specific fragments corresponding to the gene of interest;

ii. labeling each of the fragments to form individual diagnostic probes; and

iii. mixing the labeled fragments in predetermined concentrations to form a multi-gene diagnostic probe.

The multi-gene probe is used to screen a thin section of a tumor specimen. The preferred analysis technique is fluorescence in situ hybridization (FISH) assay. The resultant signal after imaging has prognostic significance. Since this multi-gene probe targets one or more independent alterations, it provides a one step and highly efficient system for breast cancer prognosis.

The present invention improves predication of disease outcome in a larger population of breast cancer patients by enabling detection of multiple changes at the molecular level that correlates with prognosis, and significantly reduces the time and cost normally required for assessment of prognostic markers in breast tumors. The present invention permits the assessment of at least five independent prognostic markers via a single step.

### BRIEF DESCRIPTION OF THE DRAWINGS

FIG. 1 shows the steps involved in the generation of gene specific probes for fluorescence in situ hybridization analysis of breast tumors. [i] Gene specific PCR primers are used to generate DNA fragments (of different sizes) unique to HER2 (1100 pb), TopoIIα (2342 bp), NM23-H1 (1785 bp), CK19 (365 bp) and MMP9 (601 bp). [ii] PCR fragments are purified by HPLC and cloned into specific restriction sites of pUC19 cloning vectors. The gene specific fragments are released after cloning using specific restriction enzymes and purified.

FIG. 2 shows steps involved in cloning of PCR generated fragments and labeling with fluorescent dyes.

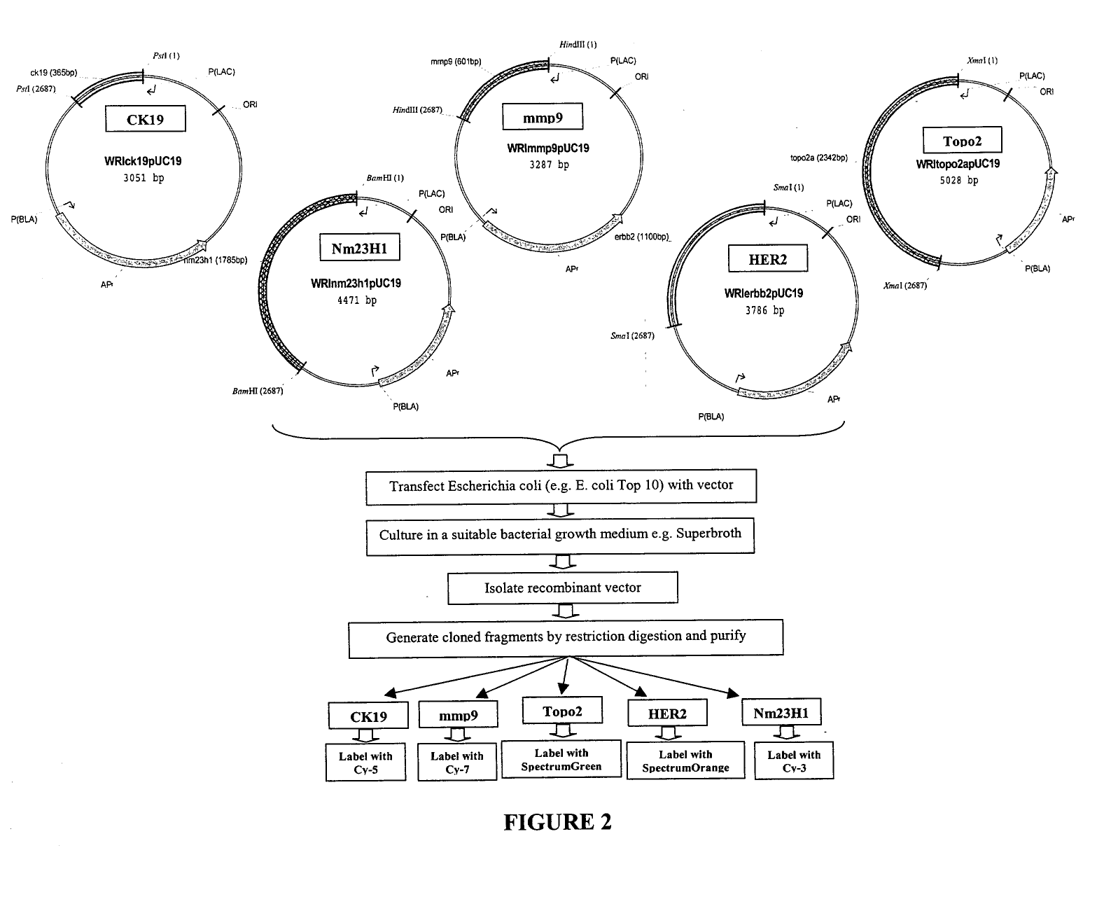

FIG. 3 shows the steps for preparation of a multi-gene probe for fluorescence in situ hybridization analysis of breast tissue.

### DETAILED DESCRIPTION OF THE INVENTION

The present invention solves the problem of inadequate broad-range prognostic factors for breast cancer by providing a multi-gene probe for a single-step determination of disease outcome. The 5-genes chosen are known to show altered expression in different breast tumors. The altered expression of any one of them has a similar prognostic significance, with respect to disease-free survival or overall survival. The multi-gene probe is labeled and used to screen a tumor specimen. The signal generated after the assay serves as a prognostic marker. Since five prognostic markers are simultaneously assessed, this strategy covers a wider variety of breast cancers and so improves disease outcome prediction in a wider population.

The five prognostic markers include HER2. HER2 is an acronym for human epidermal growth factor receptor, also known as c-erbB-2/neu. Growth factors are protein products of genes called proto-oncogenes, which are fundamentally important for normal cells. The proto-oncogenes interact with other genes and their products; these genes, called tumor suppressor genes, also have important roles in normal cell division. HER2 gene amplification and protein overexpression play a pivotal role in oncogenic transformation, tumorigenesis and metastasis. The HER2 gene (ERBB2) maps on chromosome 17q 21.1. The mRNA size is 4.5 kb. The protein expressed by HER2 is a 185-kDa tyrosine kinase receptor for heregulin and other members of the heregulin family.

Topoisomerase IIα (Topo IIα) plays a key role in DNA replication and is a target for multiple chemotherapeutic agents. In breast cancer, Topo IIα expression has been linked to cell proliferation and HER2/neu protein overexpression. Topo IIα (170 kD) maps at chromosome 17q21-q22, and encodes a protein that controls topological states of DNA.

Nm23 is an acronym for nonmetastatic protein 23 or nucleoside diphosphate (NPD) kinase-A (NDPKA). The underexpression of the NM23-H1 gene is related to cell proliferative activity. The NM23-H1 gene maps to 17q22 and consists of 5 exons and 4 introns spanning 8.5 kb. The mRNA size is 0.8 kb. The NM23-H1 gene encodes a 17 KD protein.

Cytokeratin 19 (CK19) is one of the families of genes for keratins 13, 14, 15, 16, 17, and 19 contained in less than 150 kb of genomic DNA in the region 17q21-q22. The mRNA size is 1.3 kb. The gene expresses a 40-kda acidic keratin component of intermediate filaments. CK19 protein is found on the surface of epithelial cells.

Matrix metalloproteinase-9 (MMP9) is also known as 92-kD gelatinase or Gelatinase B. It is a collagenase type IV-B (CLG4B). In highly metastatic tumor cells, there may be conspicuous expression of MMP9. The MMP9 gene (CLG4B) maps to 20 q11.2-q13.1. MMP9 has 13 exons and similar intron locations. The 13 exons of MMP9 are 3 more than have been found in other members of this gene family. The extra exons encode the amino acids of the fibronectin-like domain, which has been found only in MMP-2 and MMP-9. The mRNA size is 2.8 kb.

The present invention preferably uses polymerase chain reaction (PCR) to prepare gene-specific fragments. PCR allows an extremely large number of copies to be synthesized of any given DNA sequence provided that two oligonucleotide primers are available that hybridize to the flanking sequences on the complementary DNA strands. The reaction requires the target DNA, the two primers, all four deoxyribonucleoside triphosphates, Mg²⁺ and a thermostable DNA polymerase or enzyme. A PCR cycle is repeated for a set number of times depending on the degree of amplification required.

A PCR cycle consists of three steps:

(1) Denaturation. The reaction mixture is heated to 95° C. for a short time period to denature the target DNA into single strands that can act as templates for DNA synthesis.

(2) Primer annealing. The mixture is rapidly cooled to a defined temperature typically around 55° C., which allows the two primers to bind to the sequences on each of the two strands flanking the target DNA region. This annealing temperature is calculated to ensure that the primers bind only to the desired DNA sequences. One primer binds to each strand. The two parental strands do not reanneal with each other because the primers are in large excess over parental DNA.

(3) Elongation. The temperature of the mixture is raised to 72° C. (usually) and kept at this temperature for a pre-set period of time to allow DNA polymerase to elongate each primer by copying the single-stranded templates. Thus at the end of this incubation, both single-stranded template strands have been made partially double stranded. The new strand of each double-stranded DNA extends for a variable distance downstream.

The three steps of the PCR cycle are repeated. Thus in the second cycle, the four strands denature, bind primers and are extended. No other reactants need to be added. The three steps are repeated once more for a third cycle and so on for a set number of additional cycles. By the third cycle, some of the PCR products represent DNA sequence only between the two primer sites and the sequence does not extend beyond these sites. As more and more reaction cycles are carried out, this type of double-stranded DNA molecule becomes the majority species present.

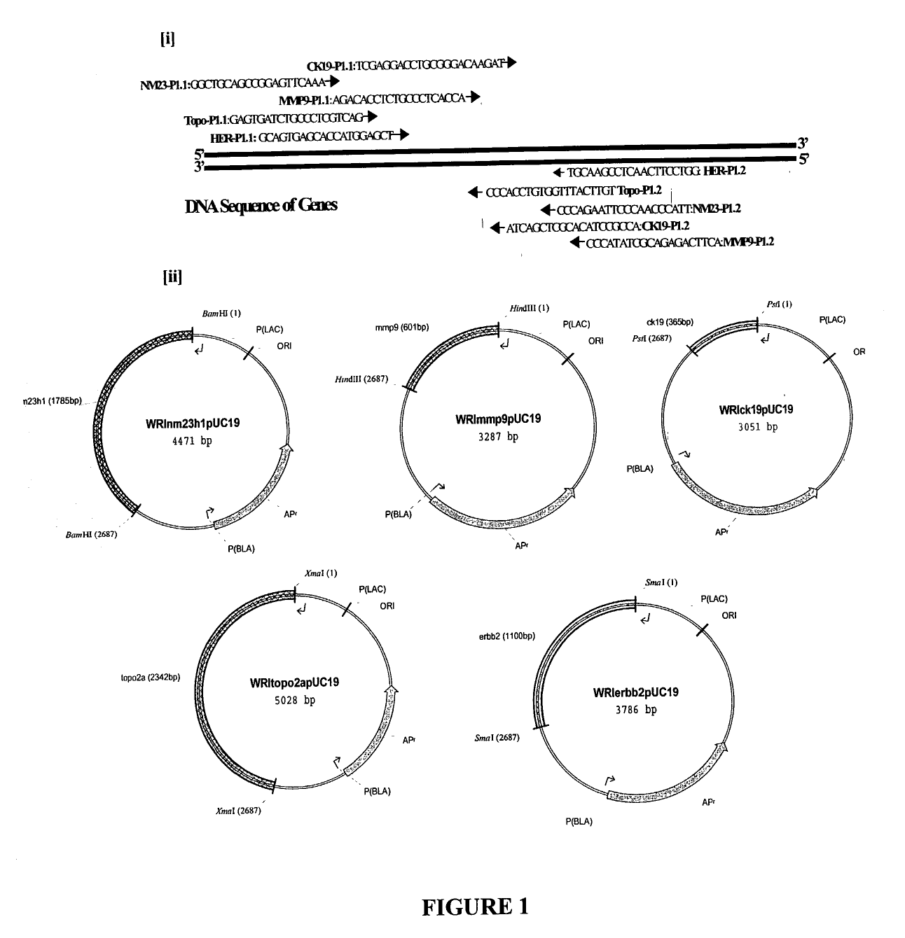

As shown in FIG. 1, sequence and gene specific primers are used to generate DNA fragments (of different sizes) unique to HER2, Topo IIα, NM23-H1, CK19 and MMP9 by PCR. The PCR generated fragments are isolated from the PCR reaction and purified.

The primers are designed to be complementary to the target DNA such that they can be extended by the DNA polymerase towards each other. Each one of a pair of PCR primers needs to be about 18-30 nt long and to have similar G+C content so they anneal to their complementary sequences at similar temperatures. Since the DNA gene sequences of the present invention are known, primer design is straightforward and may be accomplished by techniques well known in the art.

In preparing the PCR fragments, the thermostable DNA polymerase is TaqPlus Long PCR system (Stratagen Inc, La Jolla, Calif.).

The accession number for the target DNA for HER2 is nm004448 and the DNA sequence is available at http://genome.ucsc.edu. The primer sets for generation of DNA fragments for HER2 are illustrated in FIG. 1 as HER-P1.1/1.2, HER-P2.1/2.2 and HER-P3.1/3.2 HER (P1.1, 2.1 and 3.1 are the forward primers for each region and P1.2, 2.2, 3.2 are the corresponding reverse primers, respectively). HER-P 1.1 is 5′-GCAGTGAGCACCATGGAGCT-3′, HER-P 1.2 is 5′-TGCAAGCCTCAACTTCCTGG-3′, HER-P2.1 is 5′-CTCTTGGGACCTAGTCTCTG-3′, HER-P2.2 is 5′-ACACTGTTAACCATGGTCCC, HER-P3.1 is 5′-GGATTACAAGCGCCCGCTAATT-3′ and HER-P3.2 is 5′-GAGGTTTCGCTCTGTCACCC-3′.

The accession number for the target DNA for Topo IIα is nm001067 and the DNA sequence is available at http://genome.ucsc.edu. The primer sets for generation of DNA fragments for Topo IIα are illustrated in FIG. 1 as Topo-P1.1/1.2, Topo-P2.1/2.2 and Topo-P3.1/3.2. The primer for Topo-P1.1 is 5′-GAGTGATCTGCCCTCGTCAG-3′, Topo-P1.2 is 5′-CCCACCTGTGGTTTACTTGT-3′, Topo-P2.1 is 5′-GAATAGAATGTTTCCAGTAAGC-3′, Topo-P2.2 is 5′-CCTGGTTTCAAACCTTTAAA-3′, Topo-P3.1 is 5′-ATTGAGGATACTTACGTTTG-3′ and Topo-3.2 is 5′-GAGACCAAGACTGGAGATTT-3′.

The accession number for the target DNA for NM23-H1 is x73066 and the DNA sequence is available at http://genome.ucsc.edu. The primer sets for generation of DNA fragments for NM23-H1 are illustrated in FIG. 1 as NM23-P1.1/1.2, NM23-P2.1/2.2 and NM23-P3.1/3.2. The primer for NM23-P1.1 is 5′-GGCTGCAGCCGGAGTTCAAA-3′, NM23-P1.2 is 5′-CCCAGAATTCCCAACCCATT-3′, NM23-P2.1 is 5′-CCGCTTGAGACGGATGACGCTGTA-3′, NM23-P2.2 is 5′-TCCCTTGCTTCCTGCCTCCA-3′, NM23-P3.1 is 5′-ATAAAATTAGCCAAGTCTGG-3′ and NM23-P3.2 is 5′-TAATCTACCAGTTCCTCAGG-3′.

The accession number of the target DNA for CK19 is u85961.1 and the DNA sequence is available at http://www.ncbi.nlm.nih.gov. The primer set for generation of a DNA fragment for CK19 is illustrated in FIG. 1 as CK19-P1.1/1.2. The primer for CK19-P1.1 is 5′-TCGAGGACCTGCGGGACAAGAT-3′ and CK19-P1.2 is 5′-ATCAGCTCGCACATCCGCCA-3′.

The accession number of the target DNA for MMP9 is nm004994 and the DNA sequence is available at http://genome.ucsc.edu. The primer sets for generation of DNA fragments for MMP9 are illustrated in FIG. 1 as MMP9-P1.1/1.2, MMP9-P2.1/2.2, and MMP9-P3.1/3.2. The primer for MMP9-P1.1 is 5′-AGACACCTCTGCCCTCACCA-3′, MMP9-P1.2 is 5′-CCCATATCGCAGAGACTTCA-3′, MMP9-P2.1 is 5′-AGCGGCCCTCGAAGATGAAG-3′, MMP9-P2.2 is 5′-GACCTGTTTCTTCAGAGCAC-3′, MMP9-P3.1 is 5′-TGACTTCCCTTTCTTACCAG-3′ and MMP9-3.2 is 5′-CAAAGGTGAGAAGAGAGGGC-3′.

After the PCR reaction is complete, the gene specific DNA fragments are isolated from the reaction mixture and purified using DNA spin columns (Qiagen Inc., Valencia, Calif.). The fragments are then cloned into specific restriction sites (HER2=SmaI; Topo2=XmaI; NM23H1=BamHI; CK19=PstI and MMP9=HindIII) of a cloning vector (e.g., pUC19). The recombinant vector is used to transfect bacteria (e.g., Escherichia coli Top 10) and after culturing the bacteria in a suitable medium for approximately 24 hours the vector is isolated using plasmid isolation kits (Qiagen, Inc., Valencia, Calif.) and the cloned inserts generated by restriction digestion and purified (FIG. 1).

Each of the separated and purified gene-specific fragments is labeled with fluorescent dyes. Preferably, each of the PCR fragments is labeled with a different fluorescent dye as follows:

| | Fluorescent Dye | |
|---|---|---|
| Fragment | Type (Color) | Preferred Fluorescent Dye |
| HER2 | SpectrumOrange ™ | SpectrumOrange ™ (Vysis Inc.) |
| | (Orange) | |
| Topo IIα | SpectrumGreen ™ (Green) | SpectrumGreen ™ (Vysis Inc.) |
| NM23-H1 | Cy3 ™ (Red) | Cy3 ™ (Amersham Biosciences) |
| CK19 | Cy5 ™ (Blue) | Cy5 ™ (Amersham Biosciences) |
| MMP9 | Cy7 ™ (Yellow) | Cy7 ™ (Amersham Biosciences) |

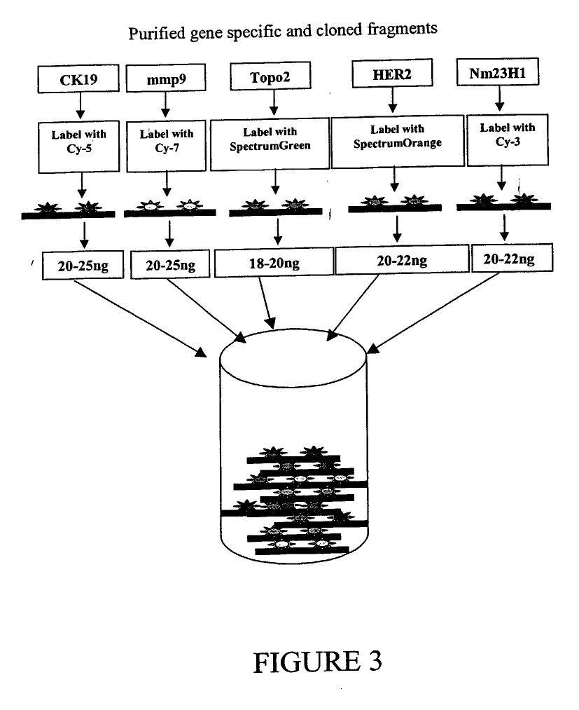

A multi-gene diagnostic probe is prepared as illustrated in FIG. 3 by mixing precise concentrations of each labeled fragment as follows:

| Fragment | General Concentration Range | Preferred Concentration |
|---|---|---|
| HER2 | 10-25 ng | 22 ng |
| Topo IIα | 10-25 ng | 18 ng |
| NM23-H1 | 10-25 ng | 20 ng |
| CK19 | 10-25 ng | 25 ng |
| MMP9 | 10-25 ng | 25 ng |

The multi-gene probe is used to screen a thin section of a tumor specimen. The tumor specimen to be screened is first fixed with, for example, formaldehyde, embedded in paraffin wax and then cut into thin sections 4-5 μm thick. The screening is preferably accomplished by in situ hybridization. More specifically, it is possible to incubate radioactive or fluorescent probes with sections of tissues, wash away excess probe and then detect where the probe has hybridized. The most preferred screening technique is by fluorescence in situ hybridization (FISH) assay.

The FISH assay broadly comprises de-paraffinization, denaturation of the specimen DNA, preparation of the probe mixture, hybridization of the specimen DNA and the probe mixture, and post-hybridization washes. Standard protocols may be used to determine the optimum denaturation time and temperature, typically 72±1° C. for 5 minutes; hybridization time and temperature, typically 37° C. for 14-18 hours; post-hybridization wash time and temperature, typically 72±1° C. for 2 minutes. The post-hybridization wash buffer composition is typically 2×SSC/0.3% NP-40.

The fluorescent fragments absorb light at an excitation (EX) wavelength and then emit it at an emission (EM) wavelength. After unbound probe is removed, the slide is air dried in the dark, a counter stain (DAPI) is applied and the specimen is illuminated at the excitation wavelength to enumerate the signal. The resultant signal after imaging will indicate the status of the DNA targeted by each probe. The DNA copy number has prognostic significance.

Signal enumeration is carried out by imaging the hybridized slides under a fluorescence microscope with filters appropriate for each probe. Use a 25× or 40× objective for an initial scan of the entire tissue section hybridized. Select an area of good nuclei distribution and switch to a 63× or 100× objective for enumeration of 60 nuclei to determine DNA copy number. Presently, the HER2 DNA copy number is best characterized in relation to the prognostic significance. For example, HER 2 gene copy number is determined with a HER2 specific probe and a chromosome 17-enumeration probe (Vysis Inc.). The ratio between the HER2 and chromosome-7 copy number has a prognostic significance. Ratios above 2.0 are typically associated with poor prognosis, characterized by shorter disease-free and overall survival in node positive patients.

The multi-gene probe may be packaged in the form of an assay kit. The kit would typically include the fluorescent-labeled multi-gene probe, control slides with human cell line specimens having different levels of HER2 gene amplification, a counter stain such as DAPI, NP-40, 20×SSC solution, 4% formalin in PBS, NaOH, protease/buffer, and microcentrifuge tubes.

While the foregoing invention has been described in some detail for purposes of clarity and understanding, it will be appreciated by one skilled in the art from a reading of this disclosure that various changes in form and detail can be made without departing from the true scope of the invention and appended claims.

We claim:

1. A method of assessing breast cancer prognosis in a human subject, comprising the steps of:
(a) obtaining a biopsy of a tumor from the breast of the human subject; and
(b) analyzing the sample with a multi-gene probe targeting the HER2, Topo IIα NM23-H1, CK19 and MMP9 DNA.

2. The method of claim 1 wherein the multi-gene probe is a 5-gene probe.

3. The method of claim 2 wherein the multi-gene probe comprises selected polymerase chain reaction generated fragments.

4. The method of claim 3 wherein each of the fragments is cloned into a vector.

5. The method of claim 4 wherein each of the fragments is released from the vector by enzymatic digestion and labeled with a different fluorescent dye.

6. The method of claim 5 wherein the labeled fragments are mixed in predetermined concentrations.

7. The method of claim 6 wherein the labeled fragments and buffer components are assembled as a easy-to-use assay kit.

8. A method of assessing breast cancer prognosis in a human subject, comprising the steps of:
(a) obtaining a sample of a thin section of tissue containing tumor from the breast of the human subject; and
(b) analyzing the sample by fluorescence in situ hybridization with a multi-gene probe comprising gene specific fragments each labeled with a fluorescent dye mixed in predetermined concentrations to simultaneously target the HER2, Topo IIα, NM23-H1, CK19 and MMP9 genes.

9. A method of assessing breast cancer prognosis in a human subject, comprising the steps of:
(a) generating DNA fragments unique to HER, Topo IIα, NM23-H1, CK19 and MMP9;
(b) isolating, purifying and cloning the generated fragments into a cloning vector;
(c) releasing the fragments by enzymatic digestion and labeling each fragment with a different fluorescent dye;
(d) mixing the labeled fragments in predetermined concentrations to form a multi-gene probe;
(e) obtaining a sample of a tumor from the breast of the human subject; and
(f) analyzing the sample with the multi-gene probe.

10. The method of claim 9 wherein the multi-gene probe is a 5-gene probe.

11. The method of claim 10 wherein the multi-gene probe comprises polymerase chain reaction fragments.

12. An assay kit for assessing breast cancer prognosis in a human subject, comprising a package containing a multi-gene probe targeting the HER2, Topo IIα, NM23-H1, CK19 and MMP9 genes.

13. The assay kit of claim 12 wherein the multi-gene probe is a 5-gene probe.

14. The assay kit of claim 13 wherein the multi-gene probe comprises polymerase chain reaction fragments.

15. The assay kit of claim 14 wherein each of the fragments is labeled with a fluorescent dye.

16. The assay kit of claim 15 wherein the labeled fragments are mixed in predetermined concentration.

17. The assay kit of claim 12 further comprising pre- and post-hybridization wash components.

18. A multi-gene probe for assessing breast cancer prognosis in a human subject, comprising:
(a) a labeled fragment complementary to at least a region of the HER2 gene sequence;
(b) a labeled fragment complementary to at least a region of the Topo IIα gene sequence;
(c) a labeled fragment complementary to at least a region of the NM23-H1 gene sequence; (d) a labeled fragment complementary to at least a region of the CK19 gene sequence; and
(e) a labeled fragment complementary to at least a region of the MMP9 gene sequence;
each of the fragments being labeled with a different fluorescent label and each of the fragments being useable to detect its complementary gene copy number by hybridization.

19. The multi-gene probe of claim 18 wherein the multi-gene probe is a 5-gene probe.

20. The multi-gene probe of claim 19 wherein the multi-gene probe comprises polymerase chain reaction fragments.

21. The multi-gene probe of claim 20 wherein each of the fragments is labeled with a fluorescent dye.


SEQUENCE LISTING (26 sequences)

SEQ ID NO: 1 - DNA, 20 bp, Artificial Sequence
  Description of Artificial Sequence Primer
gcagtgagcaccatggagct

SEQ ID NO: 2 - DNA, 20 bp, Artificial Sequence
  Description of Artificial Sequence Primer
tgcaagcctcaacttcctgg

SEQ ID NO: 3 - DNA, 20 bp, Artificial Sequence
  Description of Artificial Sequence Primer
ctcttgggacctagtctctg

SEQ ID NO: 4 - DNA, 20 bp, Artificial Sequence
  Description of Artificial Sequence Primer
acactgttaaccatggtccc

SEQ ID NO: 5 - DNA, 22 bp, Artificial Sequence
  Description of Artificial Sequence Primer
ggattacaagcgcccgctaatt

SEQ ID NO: 6 - DNA, 20 bp, Artificial Sequence
  Description of Artificial Sequence Primer
gaggtttcgctctgtcaccc

SEQ ID NO: 7 - DNA, 20 bp, Artificial Sequence
  Description of Artificial Sequence Primer
gagtgatctgccctcgtcag

SEQ ID NO: 8 - DNA, 20 bp, Artificial Sequence
  Description of Artificial Sequence Primer
cccacctgtggtttacttgt

SEQ ID NO: 9 - DNA, 22 bp, Artificial Sequence
  Description of Artificial Sequence Primer
gaatagaatgtttccagtaagc

SEQ ID NO: 10 - DNA, 20 bp, Artificial Sequence
  Description of Artificial Sequence Primer
cctggtttcaaacctttaaa

SEQ ID NO: 11 - DNA, 20 bp, Artificial Sequence
  Description of Artificial Sequence Primer
attgaggatacttacgtttg

SEQ ID NO: 12 - DNA, 20 bp, Artificial Sequence
  Description of Artificial Sequence Primer
gagaccaagactggagattt

SEQ ID NO: 13 - DNA, 20 bp, Artificial Sequence
  Description of Artificial Sequence Primer
ggctgcagccggagttcaaa

SEQ ID NO: 14 - DNA, 20 bp, Artificial Sequence
  Description of Artificial Sequence Primer
cccagaattcccaacccatt

SEQ ID NO: 15 - DNA, 24 bp, Artificial Sequence
  Description of Artificial Sequence Primer
ccgcttgagacggatgacgctgta

SEQ ID NO: 16 - DNA, 20 bp, Artificial Sequence
  Description of Artificial Sequence Primer
tcccttgcttcctgcctcca

SEQ ID NO: 17 - DNA, 20 bp, Artificial Sequence
  Description of Artificial Sequence Primer
ataaaattagccaagtctgg

SEQ ID NO: 18 - DNA, 20 bp, Artificial Sequence
  Description of Artificial Sequence Primer
taatctaccagttcctcagg

SEQ ID NO: 19 - DNA, 22 bp, Artificial Sequence
  Description of Artificial Sequence Primer
tcgaggacctgcgggacaagat

SEQ ID NO: 20 - DNA, 20 bp, Artificial Sequence
  Description of Artificial Sequence Primer
atcagctcgcacatccgcca

SEQ ID NO: 21 - DNA, 20 bp, Artificial Sequence
  Description of Artificial Sequence Primer
agacacctctgccctcacca

SEQ ID NO: 22 - DNA, 20 bp, Artificial Sequence
  Description of Artificial Sequence Primer
cccatatcgcagagacttca

SEQ ID NO: 23 - DNA, 20 bp, Artificial Sequence
  Description of Artificial Sequence Primer
agcggccctcgaagatgaag

SEQ ID NO: 24 - DNA, 20 bp, Artificial Sequence
  Description of Artificial Sequence Primer
gacctgtttcttcagagcac

SEQ ID NO: 25 - DNA, 20 bp, Artificial Sequence
  Description of Artificial Sequence Primer
tgacttccctttcttaccag

SEQ ID NO: 26 - DNA, 20 bp, Artificial Sequence
  Description of Artificial Sequence Primer
caaaggtgagaagagagggc